(19) United States
(12) Patent Application Publication
(10) Pub. No.: US 2026/0069527 A1
(43) Pub. Date: Mar. 12, 2026

(54) Anhydrous Cosmetic Composition with a Volatile Hydrocarbon Oil, Dodecamethylpentasiloxane and a Silicone Elastomer Possessing Carboxylic Acid Functions

(71) Applicant: L'OREAL, PARIS (FR)

(72) Inventors: GUILLAUME KERGOSIEN, CHEVILLY-LARUE (FR); KÉVIN DU, CHEVILLY-LARUE (FR)

(73) Assignee: L'OREAL, Paris (FR)

(21) Appl. No.: 19/145,530

(22) PCT Filed: Dec. 13, 2023
(86) PCT No.: PCT/EP2023/085663

§ 371 Date: Jul. 3, 2025

(30) Foreign Application Priority Data
Jan. 4, 2023 (FR) 2300057

(51) Int. Cl. A61K 8/893; A61K 8/31; A61K 8/58; A61K 8/898; A61Q 1/02

(52) U.S. Cl. CPC A61K 8/893; A61K 8/31; A61K 8/585; A61K 8/898; A61Q 1/02; A61K 2800/31; A61K 2800/43

(57) ABSTRACT

The present invention relates to an anhydrous composition for caring for and/or making up keratin materials, in particular the skin, comprising, preferably in a physiologically acceptable medium: a) an oily phase comprising i) at least one volatile hydrocarbon oil H1 and ii) dodecamethylpentasiloxane; and b) at least one silicone elastomer possessing carboxylic acid functions. The invention also relates to a method for coating keratin materials, more particularly for making up and/or caring for keratin materials, such as the skin, characterized in that it comprises the application to keratin materials of an emulsion as defined above.

### TECHNICAL FIELD

The present invention is targeted at providing, for the field of the care and/or make up of keratin materials, in particular of the skin, a novel anhydrous composition comprising a) an oily phase comprising i) at least one volatile hydrocarbon oil and ii) dodecamethylpentasiloxane and b) at least one silicone elastomer possessing carboxylic acid functions.

Cosmetic make-up compositions are commonly employed to give an aesthetic colour to keratin materials, such as the skin, but also to enhance the beauty of irregular skin, by making it possible to hide marks and dyschromias, and to reduce the visibility of relief imperfections, such as pores and wrinkles and also spots and acne marks.

The application of a make-up composition, such as a foundation, is the most effective approach for enhancing the beauty of the skin and reducing the visibility of relief imperfections. In this respect, coverage is one of the main properties sought.

Use is generally made, in these compositions, at high amounts, of pigments based on metal oxides, such as iron oxides and titanium oxides, in combination with particles known as fillers having a blurring or soft focus effect. However, these compositions have a tendency to accumulate in the reliefs, such as pores and wrinkles, to result in a non-uniform deposit on the skin highlighting the imperfections thereof and giving a matt finish perceived as unnatural on the skin.

Consumers are looking for products for caring for and/or making up keratin materials, in particular the skin, which exhibit a texture which is comfortable at the sensory level and on application, in particular which are easy to take up and which spread easily over the area of keratin material to be covered.

There have already been proposed, in the published documents WO2015066161 (US20160200876), WO2015066165, WO2015066199, WO2015167963, WO20180165434 (U.S. Ser. No. 10/918,587B2), IPCOM000250448D and IPCOM000250657D, make-up compositions based on silicone elastomers possessing carboxylic acid functions exhibiting good performance qualities in terms of wear property and of durability. However, these polymers generally exhibit a texture which is difficult to take up and to apply and have a tendency to give make-up compositions, the insufficiently fluid, supple and homogeneous consistency of which does not make it possible to take up and spread the product easily on the skin.

One of the objectives of this invention is to produce a care and/or make-up composition comprising at least one silicone elastomer possessing carboxylic acid functions having a consistency which is sufficiently fluid, smooth, supple and homogeneous to be taken up and easily spread on the skin.

In the course of its research studies, the Applicant Company has discovered, unexpectedly, that these objectives can be achieved with an anhydrous composition for caring for and/or making up keratin materials, in particular the skin, comprising, preferably in a physiologically acceptable medium:
a) an oily phase comprising
i) at least one volatile hydrocarbon oil H₁ and
ii) dodecamethylpentasiloxane; and
b) at least one silicone elastomer possessing carboxylic acid functions.

This discovery forms the basis of the invention.

The present invention relates to an anhydrous composition for caring for and/or making up keratin materials, in particular the skin, comprising, preferably in a physiologically acceptable medium:
a) an oily phase comprising
i) at least one volatile hydrocarbon oil H₁ and
ii) dodecamethylpentasiloxane; and
b) at least one silicone elastomer possessing carboxylic acid functions.

The invention also relates to a method for coating keratin materials, more particularly for making up and/or caring for keratin materials, such as the skin, characterized in that it comprises the application to keratin materials of an emulsion as defined above.

The invention more particularly relates to a method for making up and/or caring for the skin, characterized in that it comprises the application to the skin of a composition as defined above.

### Definitions

In the context of the present invention, the term “keratin materials” means the skin and more particularly the areas such as the face, the cheeks, the hands, the body, the legs and thighs, the area around the eyes, and the eyelids.

The term “physiologically acceptable” is understood to mean compatible with the skin and/or its superficial body growths, which exhibits a pleasant colour, odour and feel and which does not cause unacceptable discomfort (stinging or tautness) liable to dissuade the consumer from using this composition.

Within the meaning of the present invention, the term “anhydrous” is understood to mean a composition comprising a content of less than or equal to 2% by weight, preferably of less than or equal to 1% and more preferentially of less than 0.5% by weight of water, with respect to the total weight of said composition, indeed even devoid of water. If appropriate, such small amounts of water may in particular be introduced by ingredients of the composition which may contain residual amounts thereof.

### Oily Phase

The composition according to the invention for caring for and/or making up keratin materials comprises at least one oily phase comprising
i) at least one volatile hydrocarbon oil H₁ and
ii) dodecamethylpentasiloxane.

The term “oil” is understood to mean a fatty substance which is liquid at ambient temperature (25° C.) and atmospheric pressure (760 mmHg, i.e. 10⁵ Pa).

The term “oily phase” is understood to mean an organic phase which is liquid at ambient temperature (25° C.) and atmospheric pressure containing at least one oil and optionally the cosmetic additives which are soluble or miscible in said phase.

The term “hydrocarbon-based oil” refers to an oil mainly containing carbon and hydrogen atoms and possibly one or more functions chosen from hydroxyl, ester, ether and carboxylic functions.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 2.66 Pa to 40 000 Pa, in particular ranging from 2.66 Pa to 13 000 Pa and more particularly ranging from 2.66 Pa to 1300 Pa.

The term “hydrocarbon oil” is understood to mean an oil comprising mainly carbon and hydrogen atoms and optionally one or more functions chosen from hydroxyl, ester, ether or carboxylic functions.

### Hydrocarbon Oil H₁

Mention may be made, as examples of volatile hydrocarbon oil H₁ which can be used in the oily phase of the composition of the invention, of hydrocarbon oils having from 8 to 16 carbon atoms, and in particular C₈-C₁₆ isoalkanes of petroleum origin (also called isoparaffins), such as isododecane (also called 2,2,4,4,6-pentamethylheptane), isodecane or isohexadecane. Mention may also be made of the oils sold under the trade names Isopar® or Permethyl®, branched C₈-C₁₆ esters, isohexyl neopentanoate and their mixtures. Other volatile hydrocarbon oils, such as petroleum distillates, in particular those sold under the name Shell Solt by Shell, can also be used.

The volatile hydrocarbon oils H₁ which can be used in the compositions according to the invention can be chosen from volatile linear alkanes comprising from 6 to 14 carbon atoms.

Mention may be made, as examples of linear alkanes suitable for the invention, of the alkanes described in the patent applications of Cognis WO 2007/068371 or WO 2008/155059 (mixtures of different alkanes differing by at least one carbon). These alkanes are obtained from fatty alcohols, themselves obtained from coconut oil or palm oil.

Mention may be made, as examples of linear C₆-C₁₄ alkanes suitable for the invention, of n-hexane (C₆), n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and their mixtures.

Mention may in particular be made of n-dodecane (C₁₂) and n-tetradecane (C₁₄), which are sold by Sasol respectively under the references Parafol 12-97® and Parafol 14-97®, and also their mixtures.

According to another embodiment, a mixture of n-dodecane and of n-tetradecane is used. Use may in particular be made of the dodecane/tetradecane mixture in the 85/15 ratio by weight sold by Biosynthis under the reference Vegelight 1214®.

According to yet another embodiment, use is made of a mixture of volatile linear C₉-C₁₂ alkanes with the INCI name: C9-12 Alkane, such as the product sold by Biosynthis under the reference Vegelight Silk®.

According to yet another embodiment, use is made of a mixture of n-undecane (C₁₁) and of n-tridecane (C₁₃), such as those obtained in Examples 1 and 2 of Application WO 2008/155059 from Cognis and such as that sold under the trade name Cetiol Ultimate® by BASF.

According to a particularly preferred embodiment, the volatile hydrocarbon oil H₁ will be chosen from C₈-C₁₆ isoalkanes of petroleum origin, and more particularly isododecane.

According to a preferential embodiment of the invention, the volatile hydrocarbon oil(s) H₁ is (are) present in contents ranging from 50% to 100% by weight and more preferentially ranging from 80% to 100% by weight, with respect to the total weight of the oily phase.

According to a preferential embodiment of the invention, the volatile hydrocarbon oil(s) H₁ is (are) present in contents ranging from 50% to 95% by weight and more preferentially ranging from 60% to 80% by weight, with respect to the total weight of the composition.

According to a preferential embodiment of the invention, the oily phase is present in the composition in an amount ranging from 50% to 95% by weight and more preferentially from 60% to 80% by weight, with respect to the total weight of the composition.

### Dodecamethylpentasiloxane.

Dodecamethylpentasiloxane is a linear volatile silicone oil with the structure:

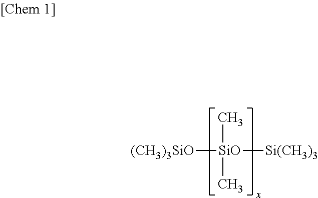

where x denotes 5.

Its mean viscosity is 2 centistokes (2 mm²/s). The method for measuring the viscosity used in the invention to characterize the silicone oils according to the invention can be the “kinematic viscosity at 25° C. Raw Product CID-012-01”, or also the “Viscosity Ubbelohde DIN 51562-1 PV04001 25° C.”.

Dodecamethylpentasiloxane can be detected in a composition containing it by gas chromatography coupled with a flame ionization detector (GC/FID), or by gas chromatography coupled with mass spectrometry (GC-MS), or by headspace gas chromatography coupled with mass spectrometry (HS-GC-MS).

It is sold in particular under the trade names KF-96L-2CS®, DM-FLUID-2CS® by Shin Etsu; BERB-DM2® by BRB International or SILICONE FLUID 2CS® by Dow Corning.

According to a preferential embodiment of the invention, the dodecamethylpentasiloxane is present in contents ranging from 1% to 50% by weight and more preferentially ranging from 1% to 10% by weight, with respect to the total weight of the oily phase.

According to a preferential embodiment of the invention, the dodecamethylpentasiloxane is present in contents ranging from 0.5% to 25% by weight and more preferentially ranging from 0.5% to 5% by weight, with respect to the total weight of the composition.

### Silicone Elastomer Possessing Carboxylic Acid Functions

The composition according to the invention for caring for and/or making up keratin materials comprises b) at least one silicone elastomer possessing carboxylic acid functions.

The term “organopolysiloxane elastomer” is understood to mean a flexible and deformable organopolysiloxane having viscoelastic properties and in particular the consistency of a sponge or of a flexible sphere. Its modulus of elasticity is such that this material withstands deformation and possesses a limited stretchability and contractability. This material is capable of regaining its original shape after stretching. In general, it is partially or completely crosslinked and is non-cyclic.

The term “silicone elastomer possessing carboxylic acid functions” is understood to mean an organopolysiloxane elastomer comprising, in its structure, at least two carboxylic acid groups with at least one —COOH function.

The composition according to the invention advantageously exhibits a content of active material of silicone elastomer possessing carboxylic acid functions ranging from 2% to 25% by weight, with respect to the weight of the composition. Preferably, this content ranges from 5% to 20% by weight, with respect to the weight of the composition.

The silicone elastomers possessing carboxylic acid functions which can be used in the compositions of the invention can be chosen from those described in the published documents WO2015066161 (US20160200876), WO2015066165, WO2015066199, WO2015167963, WO20180165434 (U.S. Ser. No. 10/918,587B2), IPCOM000250448D and IPCOM000250657D.

According to a particular embodiment, the silicone elastomer possessing carboxylic acid functions is chosen from those corresponding to the following formula (I):

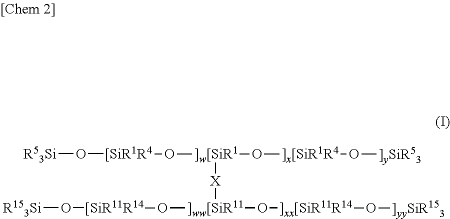

in which
the radical X corresponds to the following formula (i) or to the following formula (i′):

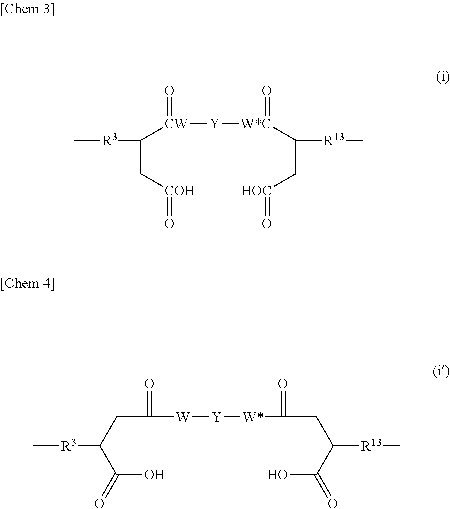

in which
W and W* independently denote an oxygen atom (O) or an N—R group where each R radical independently denotes a hydrogen atom (H) or an R¹ radical;
Y is a divalent group;
R¹, R¹¹, R⁴, R¹⁴, R⁵ and R¹⁵ independently denote a substituted or unsubstituted hydrocarbyl group;
R³ and R¹³ independently denote a divalent group;
w and ww are independently integers ranging from 0 to 1000;
x and xx are independently integers ranging from 1 to 100;
y and yy are independently integers ranging from 0 to 1000;
preferably, w and y are not simultaneously equal to 0 and ww and yy are not simultaneously equal to 0.

The term “substituted” is understood to mean that one or more hydrogen atoms of the hydrocarbyl group is (are) replaced by an atom other than hydrogen (i.e. halogen) or else that one or more carbon atoms is (are) replaced by an atom other than carbon, such as a heteroatom, for example oxygen, sulfur or nitrogen.

In the formula (I), the lower and upper portions are identical or different siloxanes. According to certain particular embodiments, each siloxane chain comprises siloxane (Si—O—Si) bonds in their respective backbones. Each siloxane chain can include siloxane bonds separated by one or more divalent groups, for example a —CH₂— linking group.

Other examples of divalent groups can include polyether groups: for example, —CH₂CH₂O— (i.e., ethylene oxide), —CH(CH₃)CH₂O— (i.e., propylene oxide) linking groups, and the like. Combinations of different divalent groups can be present in their respective backbones. Each of the divalent groups can occur just once or be repeated, i.e.: 2 times, 5 times, 10 times, indeed even >10 times, and the like. According to certain embodiments, the siloxanes do not comprise a polyether group.

In various embodiments, one or both siloxanes can comprise at least one [SiR¹R²—O—] unit (“D” or R*₂SiO_{2/2} units). Typically, each siloxane has a repetition of D units, which generally constitutes the linear portions of the siloxanes. The siloxanes also typically have terminal R*₃SiO_{1/2} units (“M” units).

In certain particular embodiments, one of the two siloxanes or both siloxanes can optionally be branched, partially branched and/or include a resin portion having a network of three-dimensional structure. In such cases, the corresponding siloxane(s) can additionally comprise R*SiO_{3/2} units (“T” units) and/or SiO_{4/2} units (“Q” units). The branched or resinous nature of the siloxane(s) can be attributed to the presence of T and/or Q units. The branched nature of a siloxane can be attributed to side groups of one or more D units. According to particular embodiments, one of the siloxanes or both siloxanes can be devoid of T units and/or of Q units. The two siloxanes can be identical or different: i.e. one is linear and the other is branched, or else both siloxanes are linear.

According to certain particular embodiments, the R¹ radicals independently denote an alkyl, aryl, alkenyl, alkaryl, or aralkyl radical. More particularly, the R¹ radicals denote an alkyl radical, preferably having from 1 to 20, from 1 to 15, from 1 to 10, from 1 to 6, from 1 to 4, or 1 or 2 carbon atoms, such as, for example, methyl, ethyl, propyl, butyl or pentyl.

More particularly, all the R¹ radicals designate a methyl group (i.e. —CH₃).

Preferably, the R³ radical is a hydrocarbylene, heterohydrocarbylene, or organoheterylene group. In particular, R³ is a (CH₂)n group where n is an integer ranging from 1 to 30, from 1 to 25, from 1 to 20, from 1 to 15, from 1 to 10, from 1 to 5, or from 1 to 3, and more particularly equal to 3.

According to various particular embodiments, each of the R⁴ and R⁵ radicals can independently denote a substituted or unsubstituted hydrocarbyl group, in particular chosen from those defined above for the R¹ radicals.

In some embodiments, each R⁴ radical independently denotes an alkyl or aryl (i.e., phenyl) group or an (R⁶O)ₘ group. When R⁴ is an (R⁶O)ₘ group, R⁶ is preferably an alkyl or aryl (i.e., phenyl) radical and m is an integer ranging from 1 to 50, from 1 to 25, from 1 to 10, from 1 to 5, or 1. The (R⁶O)ₘ group can be a polyether group (i.e.: with ethylene oxide and/or propylene oxide units). According to particular embodiments, an R⁴ radical can be a divalent group of a silicone side chain of the siloxane.

According to a particular embodiment, R⁴ can denote an anhydride group of formula (1)

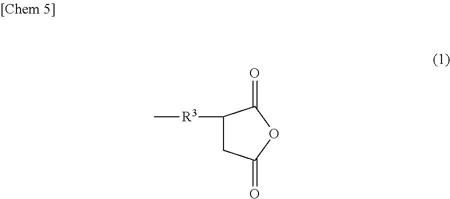

in which R³ has the same definition as indicated above.

Preferably, the R⁴ radicals denote an alkyl radical or a polyether group.

Preferably, each R⁵ radical is an R¹ radical. For example, each R⁵ and R¹ radical is an alkyl group: i.e. methyl.

Preferably, R¹¹ is identical to or different from R¹, R¹⁴ is identical to or different from R⁴, and R¹⁵ is identical to or different from R⁵. Preferentially, the R⁵ radicals denote R¹ and/or the R¹⁵ radicals denote R¹¹.

Preferably, w is an integer ranging from 0 to 1000, from 0 to 950, from 0 to 750, from 0 to 500, from 0 to 400, from 1 to 350, from 1 to 300, from 25 to 250, from 50 to 200, from 50 to 150, from 75 to 125, from 90 to 110, from 90 to 100, from 90 to 95, and more particularly is equal to 93.

Preferably, x is an integer from 1 to 100, from 1 to 75, from 1 to 50, from 1 to 25, from 1 to 20, from 1 to 10, or from 1 to 5, and more particularly is equal to 3.

Preferably, y is an integer ranging from 0 to 1000, from 0 to 950, from 0 to 750, from 0 to 500, from 0 to 400, from 1 to 350, from 1 to 300, from 1 to 250, from 1 to 200, from 1 to 150, from 1 to 100, from 1 to 75, from 1 to 50, from 1 to 25, from 1 to 20, from 1 to 15, from 1 to 10 or from 1 to 5.

According to various embodiments, w and y are not simultaneously equal to 0. In certain embodiments, the sum w+x+y varies from 25 to 1500, from 25 to 1000, from 25 to 900, from 25 to 800, from 25 to 700, from 25 to 600, from 25 to 500, from 25 to 400, from 25 to 300, from 50 to 200, from 75 to 150, from 85 to 125 or from 90 to 110.

In certain embodiments, x is at least equal to 1, at least equal to 10, at least equal to 25, at least equal to 50, at least equal to 75 or at least equal to 5.

According to particular embodiments, ww can be identical to or different from w, xx can be identical to or different from x and yy can be identical to or different from y. The units of the formula (I), in particular those represented between brackets with the subscripts w, ww, x, xx, y or yy, can be present in any order, randomly or in block form.

According to certain particular embodiments, the R¹¹ radicals, which are identical or different, denote a linear, branched or cyclic C₁-C₂₀, C₁-C₁₅, C₁-C₁₀, C₁-C₆, C₁-C₄ or C₁-C₂ alkyl radical, such as methyl, ethyl, propyl, butyl, pentyl, or the like, groups. More preferentially, the R¹¹ radicals are a methyl group.

According to certain specific embodiments, the R¹⁴ radicals, which may be identical or different, denote an alkyl or aryl (i.e. phenyl) radical or (R¹⁶O)ₘₘ. When R¹⁴ is an (R¹⁶O)ₘₘ group, R¹⁶ denotes an alkyl or aryl (i.e. phenyl) radical and mm is an integer ranging from 1 to 50, 1 to 25, 1 to 10, 1 to 5, or 1. The (R¹⁶O)ₘₘ group can be a polyether group (i.e.: with ethylene oxide and/or propylene oxide units).

According to specific embodiments, the R¹⁴ radicals independently denote an alkyl radical having from 2 to 20, from 2 to 15, from 2 to 10, from 2 to 5, or 2 carbon atoms.

According to other particular embodiments, R¹⁴ can be a divalent linking group of a silicone side chain of the siloxane.

According to a particular embodiment, R¹⁴ can denote an anhydride group of formula (1)

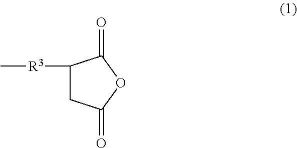

in which R³ has the same definition as indicated above.

Preferably, the R¹⁴ radicals denote an alkyl radical or a polyether group.

Preferably, ww is integer ranging from 0 to 1000, from 0 to 950, from 0 to 750, from 0 to 500, from 0 to 400, from 1 to 350, from 1 to 300, from 25 to 250, from 50 to 200, from 50 to 150, from 75 to 125, from 90 to 110, from 90 to 100, from 90 to 95, and more particularly 93.

Preferably, xx is an integer ranging from 1 to 100, from 1 to 75, from 1 to 50, from 1 to 25, from 1 to 20, from 1 to 10 or from 1 to 5, and more particularly is 3.

Preferably, yy is an integer ranging from 0 to 1000, from 0 to 950, from 0 to 750, from 0 to 500, from 0 to 400, from 1 to 350, from 1 to 300, from 1 to 250, from 1 to 200, from 1 to 150, from 1 to 100, from 1 to 75, from 1 to 50, from 1 to 25, from 1 to 20, from 1 to 15, from 1 to 10 or from 1 to 5.

In various embodiments, ww and yy are not simultaneously equal to 0. According to certain particular embodiments, the sum ww+xx+yy varies from 25 to 1500, from 25 to 1000, from 25 to 900, from 25 to 800, from 25 to 700, from 25 to 600, from 25 to 500, from 25 to 400, from 25 to 300, from 50 to 200, from 75 to 150, from 85 to 125 or from 90 to 110.

According to certain particular embodiments, xx is at least equal to 1, at least equal to 10, at least equal to 25, at least equal to 50, at least equal to 75 or at least equal to 85.

The portion of the middle X of the formula (I) corresponds to the following formula (i) or to the following formula (i′):

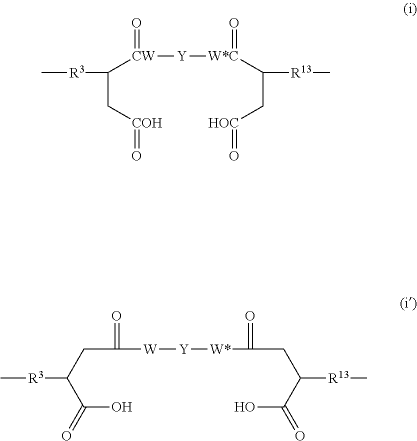

in which
each W and W* group independently denotes an oxygen atom (O) or an N—R group, with R denoting a hydrogen atom (H) or an R¹ radical; preferably, R is H;
R¹³ is a divalent group, preferably is a hydrocarbylene, a heterohydrocarbylene or an organoheterylene; in particular, R¹³ denotes a (CH₂)ₙₙ radical where nn is an integer from 1 to 30, from 1 to 25, from 1 to 20, from 1 to 15, from 1 to 10, from 1 to 5, from 1 to 3 and more particularly is 3; it being possible for R¹³ to be identical or different with respect to R³;
each Y radical is a divalent group, in particular denotes a hydrocarbylene, heterohydrocarbylene or organoheterylene radical; in particular, Y is a hydrocarbylene group having from 1 to 50, from 1 to 40, from 1 to 30, from 1 to 20, from 1 to 10, from 1 to 5, from 1 to 2 carbon atoms or can denote a group of following formula (ii):

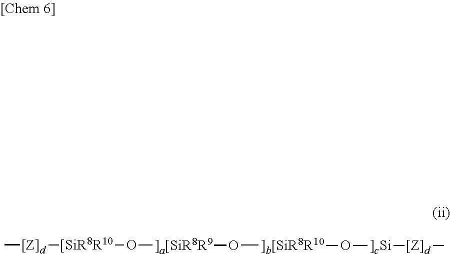

in which
   each R⁸, R⁹ independently denote a substituted or unsubstituted hydrocarbyl group, in particular chosen from an alkyl (i.e. methyl) group, an aryl (i.e. phenyl) group or a polyether group (i.e. with ethylene oxide and/or propylene oxide units);
   each R¹⁰ radical independently denotes a substituted or unsubstituted hydrocarbyl group, such as the radicals defined above for R¹ and R⁴; in particular, R¹⁰ can be an alkyl group having from 1 to 20 carbon atoms or a polyether group or a divalent linking group of a silicone side chain of the siloxane;
   each Z radical independently comprises at least one hydrocarbylene, heterohydrocarbylene or organoheterylene group, in particular is a hydrocarbylene group having from 1 to 20, from 1 to 10, from 1 to 5, or 1 or 2 carbon atom(s); in particular, Z can include siloxane bonds separated by one or more divalent groups, for example a —CH₂— linking group; other examples of divalent groups can include polyether groups: for example —CH₂CH₂O— (i.e., ethylene oxide) or —CH(CH₃)CH₂O— (i.e., propylene oxide) linking groups;
   a is an integer ranging from 0 to 1000, from 0 to 950, from 0 to 750, from 0 to 500, from 0 to 400, from 0 to 300, from 0 to 200, from 0 to 100, from 0 to 75, from 0 to 50, from 0 to 25, from 0 to 20, or
   b is an integer ranging from 1 to 1000, from 1 to 950, from 1 to 750, from 1 to 500, from 1 to 400, from 1 to 300 or from 1 to 200,
   c is an integer ranging from 0 to 1000, from 0 to 950, from 0 to 750, from 0 to 500, from 0 to 400, from 0 to 300, from 0 to 200, from 0 to 100;
   each d is equal to 0 or 1. In particular, at least one of the subscripts d is equal to 1 or both are equal to 1;
the units of the formula (ii) represented between brackets with the subscripts a, b or c being present in any order, randomly or in block form.

According to a specific form, the siloxane of formula (ii) can be a resin of formula R*ₛSiO_{(4-s)/2}. Preferably, a silicone resin has, along its chain, T and/or Q units with M units and optionally D units. The R* radicals can be chosen independently from a substituted (i.e.: hydroxyl, amine functions) or unsubstituted hydrocarbonyl group and s varies from 0 to 3. The R* groups which can be used can be those described above for the R⁸, R⁹, and R¹⁰ radicals. Various combinations of such groups can be present. According to these particular cases, the silicone resin comprises a combination of M, D, T and/or Q units. It can in particular be a resin of the MDT type, a resin of the MT type, a resin of the MDQ type, a resin of the MQ type or a resin of the MDTQ type. Each of the M, D, and T units can contain different R* groups. The resin can exhibit various molecular weights, such as a number-average molecular weight ranging from 800 to 500 000.

According to a particular form of the invention, the silicone elastomers possessing carboxylic acid functions have a carboxyl equivalent ranging from 100 to 50 000, from 500 to 10 000 or from 500 to 5000.

The silicone elastomers possessing carboxylic functions of formula (I) can be obtained according to one of the synthesis processes described in the document WO2015066161 (US20160200876).

According to a first alternative form of the synthesis process, certain silicone elastomers possessing carboxylic acid functions can be obtained by reaction 1) of a first siloxane carrying pendent anhydride groups, 2) of a second siloxane carrying pendent anhydride groups and 3) of an organic polyol. According to a particular embodiment, the 3 preceding reactants can be reacted with one or more siloxanes different from the first two.

According to a second alternative form of the synthesis process, certain silicone elastomers possessing carboxylic acid functions can be obtained by reaction 1) of a first siloxane carrying pendent anhydride groups, 2) of a second siloxane carrying pendent anhydride groups and 3) of an organic polyamine. According to a particular embodiment, the 3 preceding reactants can be reacted with one or more additional siloxanes different from the first two.

According to a third alternative form of the synthesis process, certain silicone elastomers possessing carboxylic acid functions can be obtained by reaction 1) of a first siloxane carrying pendent anhydride groups, 2) of a second siloxane carrying pendent anhydride groups and 3) of a third siloxane having at least two hydroxyl groups. According to a particular embodiment, the 3 preceding reactants can be reacted with one or more additional siloxanes different from the first two.

According to a fourth alternative form of the synthesis process, certain silicone elastomers possessing carboxylic acid functions can be obtained by reaction 1) of a first siloxane carrying pendent anhydride groups, 2) of a second siloxane carrying pendent anhydride groups and 3) of a third siloxane having pendent amine groups and/or terminal amine groups. According to a particular embodiment, the 3 preceding reactants can be reacted with one or more additional siloxanes different from the first two.

According to a fifth alternative form of the synthesis process, certain silicone elastomers possessing carboxylic acid functions can be obtained by reaction 1) of a first siloxane carrying pendent hydroxyl groups, 2) of a second siloxane carrying pendent hydroxyl groups and 3) of a third siloxane having at least two terminal anhydride groups. According to a particular embodiment, the 3 preceding reactants can be reacted with one or more additional siloxanes different from the first two.

According to a sixth alternative form of the synthesis process, certain silicone elastomers possessing carboxylic acid functions can be obtained by reaction 1) of a first siloxane carrying pendent amine groups, 2) of a second siloxane carrying pendent amine groups and 3) of a third siloxane having at least two terminal anhydride groups. According to a particular embodiment, the 3 preceding reactants can be reacted with one or more additional siloxanes different from the first two.

According to a seventh alternative form of the synthesis process, certain silicone elastomers possessing carboxylic acid functions can be obtained by reaction 1) of a first organic alcohol, 2) of a second organic alcohol and 3) of a siloxane having terminal anhydride groups. According to a particular embodiment, the 3 preceding reactants can be reacted with one or more additional organic alcohols different from the first two.

Mention may be made, among the silicone elastomers possessing carboxylic acid functions of formula (I), of the following compounds 1 to 12 as described respectively in Examples 4, 5 and 7 to 16 of Application WO2015066161:

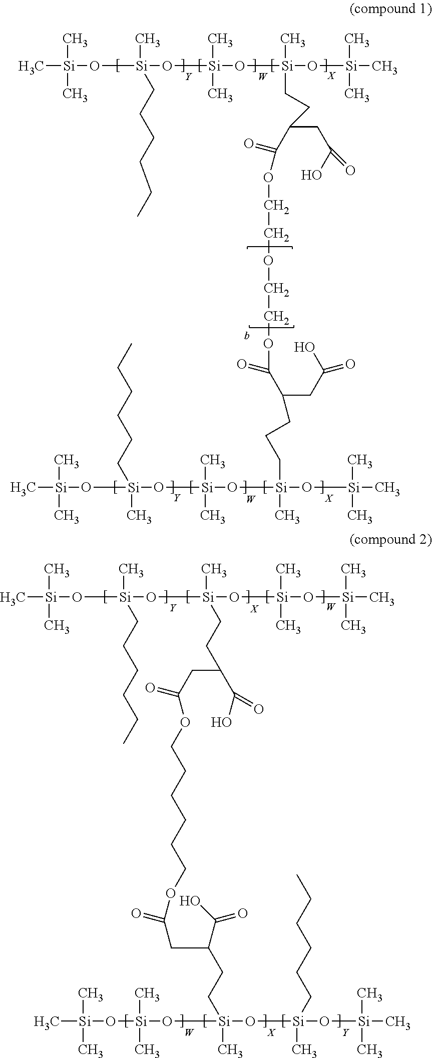

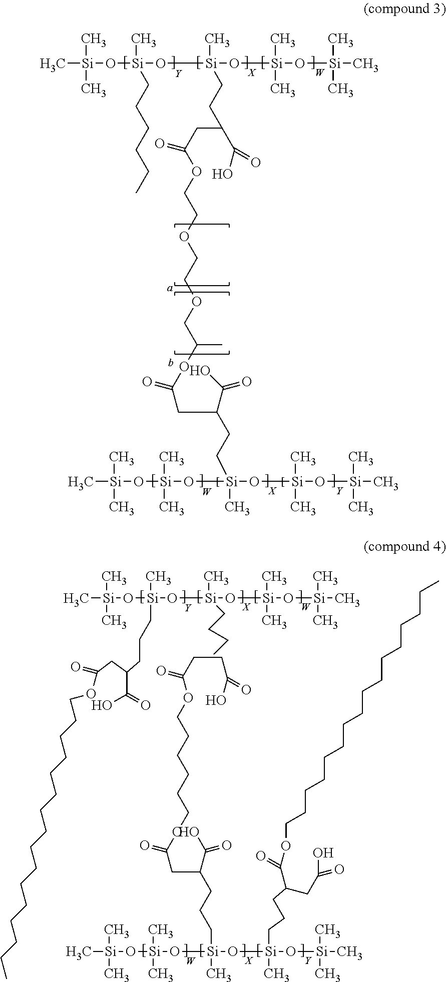

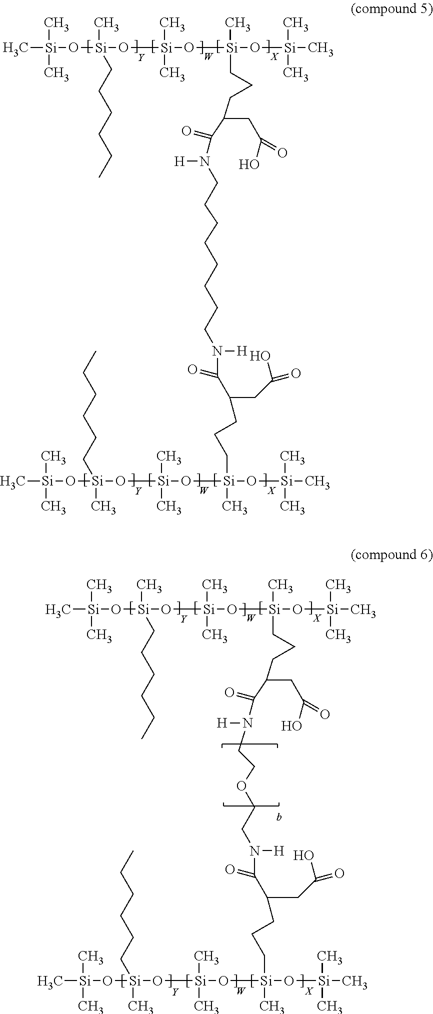

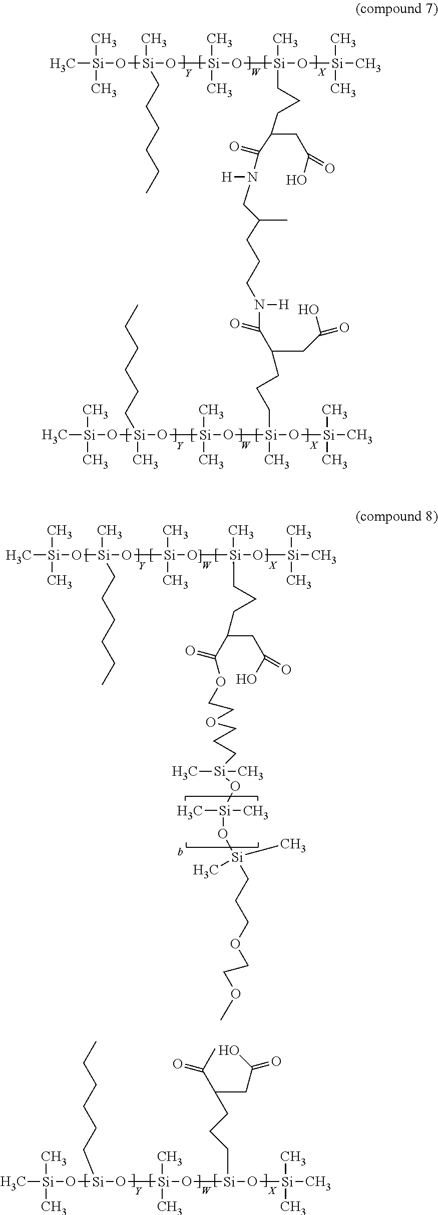

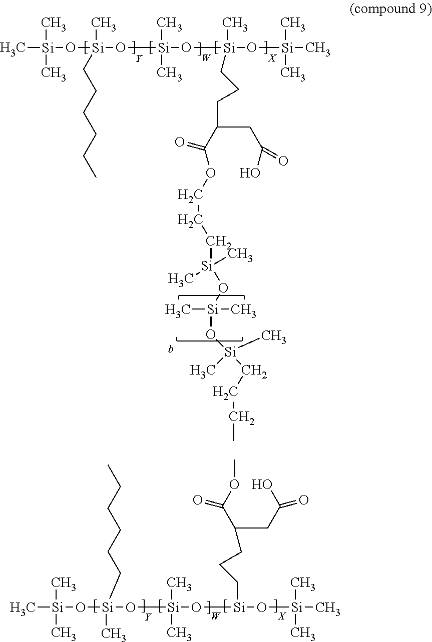

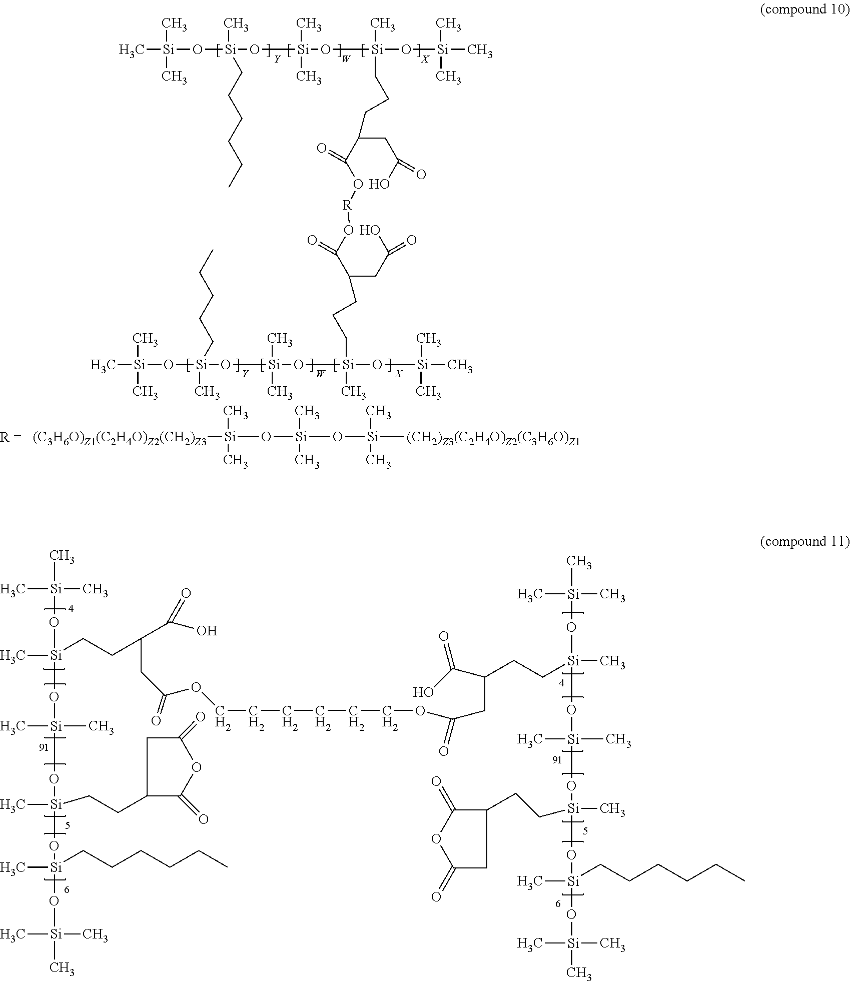

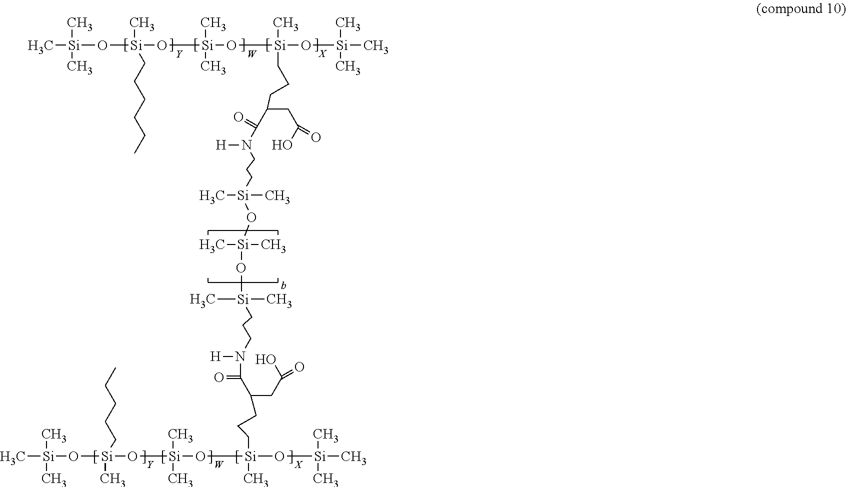

According to a particular form, the composition of the invention contains at least the compound 10 or else the compound 11 as defined above. The compound is described in particular in the document US20160200876 (compound of Example 15) and the examples of patent U.S. Ser. No. 10/918,587B2.

Mention may also be made, among the silicone elastomers possessing carboxylic acid functions which can be used in the compositions of the invention, of the silicone elastomer with the INCI name: Hexyl/Succinyl Dimethicone Crosspolymer.

According to a particular form, the silicone elastomers possessing carboxylic acid functions are in solid form. They can in particular be chosen from the compounds 2 and 3 as defined above.

According to another particular form, the silicone elastomers possessing carboxylic acid functions are in dispersion in at least one oil H₂ and are provided in gel form.

The oil H₂ can be chosen from volatile oils and/or non-volatile oils.

The term “non-volatile oil” is understood to mean an oil which remains on the keratin material at ambient temperature (25° C.) and atmospheric pressure (760 mmHg) for at least several hours and which has in particular a vapour pressure of less than 10⁻³ mmHg (0.13 Pa).

The oil(s) H₂ can be chosen from hydrocarbon oils, silicone oils and their mixtures.

Within the meaning of the present invention, the term “silicone oil” is understood to mean an oil comprising at least one Si—O group, and more particularly an organopolysiloxane.

Mention may be made, among the volatile silicone oils H₂ which can be used in the compositions, for example, of volatile linear or cyclic silicone oils, in particular those having a viscosity of less than or equal to 8 centistokes (8×10⁻⁶ m²/s) and having in particular from 2 to 7 silicon atoms, these silicones optionally comprising alkyl or alkoxy groups having from 1 to 10 carbon atoms.

Mention may be made, as linear volatile silicone oil H₂ which can be used in the invention, of octamethyltrisiloxane, decamethyltetrasiloxane, dodecamethylpentasiloxane and their mixtures.

Mention may be made, as cyclic volatile silicone oil H₂ which can be used in the invention, of cyclomethicones, such as octamethylcyclotetrasiloxane, decamethylcyclopentasiloxane, dodecamethylcyclohexasiloxane and their mixtures.

Mention may be made, as examples of linear non-volatile silicone oil H₂, of polydimethylsiloxanes (also known as dimethicones); alkyl dimethicones; vinylmethylmethicones and polydimethylsiloxanes modified by aliphatic groups and/or functional groups, such as hydroxyl, thiol, carboxylic acid and/or amine groups.

Mention may be made, as examples of linear non-volatile hydrocarbon oil, of:
synthetic esters, such as oils of formula R₁COOR₂, in which R₁ represents a residue of a linear or branched fatty acid comprising from 1 to 40 carbon atoms and R₂ represents a hydrocarbon chain, in particular a branched hydrocarbon chain, containing from 1 to 40 carbon atoms, provided that the sum of the number of carbon atoms of the chains R₁ and R₂ is greater than or equal to 10; the esters can be chosen in particular from esters of fatty acid and alcohol, such as, for example, cetostearyl octanoate, esters of isopropyl alcohol, such as isopropyl myristate or isopropyl palmitate, ethyl palmitate, 2-ethylhexyl palmitate, isopropyl stearate or isostearate,
isostearyl isostearate, octyl stearate, hydroxylated esters, such as isostearyl lactate or octyl hydroxystearate, diisopropyl adipate,
heptanoates, and in particular isostearyl heptanoate,
octanoates, decanoates or ricinoleates of alcohols or of polyalcohols, such as propylene glycol dioctanoate, cetyl octanoate, tridecyl octanoate, 2-ethylhexyl palmitate and PEG-4 diheptanoate, alkyl benzoate, polyethylene glycol diheptanoate, propylene glycol di(2-ethylhexanoate),
and their mixtures, C₁₂-C₁₅ alkyl benzoates, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, hydroxylated esters, such as isostearyl lactate or diisostearyl malate; and more particularly isodecyl neopentanoate;
dialkyl carbonates, it being possible for the 2 alkyl chains to be identical or different, such as dicaprylyl carbonate, sold under the name Cetiol CC® by Cognis;
synthetic ethers having from 10 to 40 carbon atoms, such as dicaprylyl ether.

According to a preferred form of the invention, the oil(s) H₂ can be chosen from the volatile hydrocarbon oils as described above for the oils H₁. The oil(s) H₂ can be identical to or different from the oil(s) H₁ present in the oily phase of the composition.

According to a particular form of the invention, the oil(s) H₂ present in the oily dispersion of silicone elastomer possessing carboxylic acid functions is (are) identical to the oil(s) H₁ present in the oily phase of the composition.

According to a more particularly preferred form of the invention, the oil H₂ present in the oily dispersion of silicone elastomer possessing carboxylic acid functions and the oil H₁ present in the oily phase of the composition denote a C₈-C₁₆ isoalkane of petroleum origin, and more particularly isododecane.

According to a preferred form, the oily dispersion of silicone elastomer possessing carboxylic acid functions comprises from 10% to 40% by weight of silicone elastomer active material, and more preferentially from 20% to 35% by weight, with respect to the total weight of the oily dispersion.

According to a preferred form, the composition of the invention comprises at least one silicone elastomer possessing carboxylic acid functions in dispersion in isododecane and in gel form. In particular, said silicon elastomer is chosen from the compounds 1 and 4 to 11 as defined above.

According to a particularly preferred form, the composition of the invention comprises the compound 10 or the compound 11 in dispersion in isododecane and in gel form, and more particularly in a content of active material equal to 30% by weight, with respect to the total weight of the dispersion.

According to another particularly preferred form, the composition of the invention comprises a silicone elastomer possessing carboxylic acid functions in dispersion in isododecane and in gel form with the INCI name: Isododecane (and) Hexyl/Succinyl Dimethicone Crosspolymer and more particularly in a content of active material equal to 30% by weight, with respect to the total weight of the dispersion, such as the commercial product Dowsil EL-7314 Silicone Elastomer Blend® sold by Dow Corning, comprising 30% by weight as active material of said silicone elastomer.

### Pulverulent Colorants

According to a particular form of the invention, the composition according to the invention additionally comprises at least one pulverulent colorant.

The pulverulent colorants can be chosen from inorganic pigments, organic pigments, pearlescent agents and their mixtures.

The term “pigments” is understood to mean white or coloured and inorganic or organic particles which are insoluble in an aqueous medium and which are intended to colour and/or opacify the composition and/or the resulting deposit. These pigments can be white or coloured and inorganic and/or organic.

According to a particular embodiment, the pigments used according to the invention are chosen from inorganic pigments.

The term “inorganic pigment” is understood to mean any pigment which corresponds to the definition of Ullmann's Encyclopedia in the chapter “Pigments, Inorganic”. Mention may be made, among the inorganic pigments of use in the present invention, of zirconium oxide or cerium oxide, and also zinc oxide, iron oxide (black, yellow or red) or chromium oxide, manganese violet, ultramarine blue, chromium hydrate and ferric blue, titanium dioxide and metal powders, such as aluminium powder and copper powder. The following inorganic pigments can also be used: Ta₂O₅, Ti₃O₅, Ti₂O₃, TiO, ZrO₂ as a mixture with TiO₂, ZrO₂, Nb₂O₅, CeO₂, ZnS.

The size of the pigment of use in the context of the present invention is generally greater than 100 nm and can range up to 10 μm, preferably from 200 nm to 5 μm and more preferentially from 300 nm to 1 μm.

According to a particular form of the invention, the pigments exhibit a size characterized by a D[50] of greater than 100 nm and which can range up to 10 μm, preferably from 200 nm to 5 μm and more preferentially from 300 nm to 1 μm.

The sizes are measured by static light scattering using a commercial particle size analyser of MasterSizer 3000® type from Malvern, which makes it possible to determine the particle size distribution of all of the particles over a wide range which can extend from 0.01 μm to 1000 μm. The data are processed on the basis of the standard Mie scattering theory. This theory is the most suitable for size distributions ranging from the submicronic to the multimicronic; it makes it possible to determine an “effective” particle diameter. This theory is in particular described in the publication by Van de Hulst, H. C., Light Scattering by Small Particles, Chapters 9 and 10, Wiley, New York, 1957.

D[50]represents the maximum size exhibited by 50% by volume of the particles.

According to a particular form of the invention, the inorganic pigment comprises a lipophilic or hydrophobic coating.

According to a particular embodiment of the invention, the pigments can be coated according to the invention with at least one compound chosen from metal soaps; N-acylamino acids or their salts; lecithin and its derivatives; isopropyl titanium triisostearate; isostearyl sebacate; natural vegetable or animal waxes; polar synthetic waxes; fatty esters; phospholipids; silicone surface agents, such as organopolysiloxanes, alkylalkoxysilanes, such as triethoxycaprylsilane; fluorinated surface agents, such as perfluoroalkyl phosphates, perfluoropolyethers, polytetrafluoropolyethylenes (PTFE), perfluoroalkanes, perfluoroalkyl silazanes, polyhexafluoropropylene oxides, polyorganosiloxanes comprising perfluoroalkyl perfluoropolyether groups; fluorosilicone surface agents, such as perfluoroalkyl dimethicones, perfluoroalkylsilanes and perfluoroalkyltrialkoxysilanes; and their mixtures.

According to a preferential embodiment, the pigments can be coated according to the invention with an N-acylamino acid or one of its salts which can comprise an acyl group having from 8 to 22 carbon atoms, such as, for example, a 2-ethylhexanoyl, caproyl, lauroyl, myristoyl, palmitoyl, stearoyl or cocoyl group.

The amino acid can, for example, be lysine, glutamic acid or alanine. The salts of these compounds can be the aluminium, magnesium, calcium, zirconium, zinc, sodium or potassium salts. Thus, according to a particularly preferred embodiment, the pigments can be coated with an N-acylamino acid derivative which can in particular be a glutamic acid derivative and/or one of its salts, and more particularly a stearoyl glutamate, such as, for example, aluminium stearoyl glutamate.

Mention may be made, as examples of pigments treated with aluminium stearoyl glutamate, of titanium dioxide pigments and black, red and yellow iron oxide pigments sold under the trade name Nai® by Miyoshi Kasei.

According to a preferential embodiment, the pigments can be coated according to the invention with isopropyl titanium triisostearate. Mention may be made, as examples of pigments treated with isopropyl titanium triisostearate (ITT), of those sold under the trade names BWBO-12® (Iron Oxide C177499 and Isopropyl Titanium Triisostearate), BWYO-12® (Iron Oxide C177492 and Isopropyl Titanium Triisostearate) and BWRO-12® (Iron Oxide C177491 and Isopropyl Titanium Triisostearate) by Kobo.

The pigments which can be used according to the invention can also be organic pigments.

The term “organic pigment” is understood to mean any pigment which corresponds to the definition of Ullmann's Encyclopedia in the chapter, “Pigments, Organic”. The organic pigment can in particular be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) can be chosen, for example, from carmine, carbon black, aniline black, melanin, azo yellow, quinacridone, phthalocyanine blue, sorghum red, the blue pigments codified in the Colour Index under the references CI 42090, 69800, 69825, 73000, 74100 and 74160, the yellow pigments codified in the Colour Index under the references CI 11680, 11710, 15985, 19140, 20040, 21100, 21108, 47000 and 47005, the green pigments codified in the Colour Index under the references CI 61565, 61570 and 74260, the orange pigments codified in the Colour Index under the references CI 11725, 15510, 45370 and 71105, the red pigments codified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in Patent FR2 679 771.

These pigments can also be in the form of composite pigments as are described in Patent EP 1 184 426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Mention may be made, among the organic dyes, of cochineal carmine.

Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 1 O (CI 77 002), D&C Green 3 (CI 42 053), D&C Blue 1 (CI 42090).

Mention may be made, as an example of a lake, of the product known under the name D&C Red 7 (CI 15 850:1).

Preferably, the composition according to the invention comprises at least one pulverulent colorant of inorganic pigment type, in particular chosen from metal oxides, and more particularly chosen from titanium dioxides or iron oxides, which are or not coated, and their mixtures.

The pearlescent agents can be chosen from white pearlescent pigments, such as mica covered with titanium or with bismuth oxychloride, coloured pearlescent pigments, such as titanium oxide-coated mica with iron oxides, titanium oxide-coated mica in particular with ferric blue or chromium oxide, or titanium oxide-coated mica with an organic pigment of the abovementioned type, and also pearlescent pigments based on bismuth oxychloride.

Preferably, the pulverulent colorant(s) is (are) present in the composition in a content ranging from 5% to 40% by weight, more preferably from 10% to 30% by weight, more particularly from 15% to 25% by weight, with respect to the total weight of the composition.

### Additives

The compositions according to the invention can additionally comprise additives commonly used in care and/or make-up products, such as UV sunscreens, C₂-C₄ monoalcohols, such as ethanol, moisturizing agents, such as polyols, for example glycerol, propanediol or pentylene glycol, fat-soluble colorants; lipophilic thickening or gelling agents; preservatives, fragrances and their mixtures.

### Fat-Soluble Colorants

A composition according to the invention can additionally comprise at least one fat-soluble colorant and preferably in a proportion of at least 0.01% by weight, with respect to the total weight of the composition.

Within the meaning of the invention, the term “fat-soluble colorant” is understood to mean any natural or synthetic, generally organic, compound which is soluble in an oily phase or solvents miscible with a fatty substance and which is capable of imparting colour.

For obvious reasons, this amount is liable to vary significantly with regard to the desired intensity of the colour effect and to the colour intensity provided by the colorants under consideration, and its adjustment clearly falls within the competence of a person skilled in the art.

Mention may in particular be made, as fat-soluble dyes suitable for the invention, of synthetic or natural fat-soluble dyes, such as, for example, DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto or curcumin.

### Cosmetic Compositions

The present invention also relates to a cosmetic composition comprising, in a physiologically acceptable medium, a composition as defined above.

The term “physiologically acceptable medium” is understood to denote a medium which is particularly suitable for the application of a composition of the invention to the skin.

The physiologically acceptable medium is generally suited to the nature of the support onto which the composition has to be applied, and also to the appearance under which the composition has to be packaged.

### Applications

According to one embodiment, a composition of the invention can advantageously be provided in the form of a composition for caring for the skin of the body or of the face, in particular of the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention can advantageously be provided in the form of a base composition for make-up.

A composition of the invention can advantageously be provided in the form of a foundation.

Such compositions are in particular prepared according to the general knowledge of a person skilled in the art.

Throughout the description, including the claims, the expression “comprising one” should be understood as being synonymous with “comprising at least one”.

The invention is illustrated in greater detail by the examples and figures presented below. Unless otherwise indicated, the amounts indicated are expressed as percentages by weight.

### EXAMPLE 1 (INVENTION) AND EXAMPLES 2 TO 4 (OUTSIDE THE INVENTION)

The following compositions were prepared.

TABLE 1

| | | Ex. 2 | Ex. 3 | Ex. 4 |
|---|---|---|---|---|
| | Ex. 1 | outside the | outside the | outside the |
| Ingredients | invention | invention | invention | invention |
| Isododecane (and) Hexyl/Succinyl | 62.5 (i.e. 18.75% | 62.5 (i.e. 18.75% | 62.5 (i.e. 18.75% | 62.5 (i.e. 18.75% |
| Dimethicone Crosspolymer | as dry matter of | as dry matter of | as active | as active |
| (Dowsil EL-7314 Silicone Elastomer | polymer) | polymer) | material of | material of |
| Blend ®/Dow Corning in the form | | | polymer) | polymer) |
| of a gel in dispersion at 30% by | | | | |
| weight in isododecane) | | | | |
| Dodecamethylpentasiloxane 2 cst | 2.5 | 0 | 0 | 0 |
| Cyclopentasiloxane | 28.3 | 0 | 2.5 | 0 |
| Dimethicone 6 cst | 28.3 | 0 | 0 | 2.5 |
| Isododecane | qs for 100 | qs for 100 | qs for 100 | qs for 100 |

### Procedure

The formulations 1 to 10 were produced with a reactor of Trilab® or Olsa Minilab® type from VMI. An ice bath was placed under the beaker in order to retain a manufacturing temperature of less than 20° C.

The silicone elastomer was pre-dispersed under shearing. The rest of the ingredients were subsequently gradually added to the elastomer until a homogeneous composition was obtained. The formula was subsequently rendered free from bubbles under a vacuum bell.

### Rheological Analyses

The rheological measurements were carried out on each of Examples 1 to 7 in a controlled stress rheometer (RheoStress 600®, Haake). The measurements of shear stress and of rate gradient were carried out using a cone sensor (C35/2° Ti L; 222-1871; 35 mm diameter, 2° angle), with a 0.105 mm gap and a measuring plate (MPC 35; 222-1549; 35 mm diameter, sand blasted).

The temperature of the gap was controlled using an Eheim Pro 3600/2075020 hydraulic circulation pump and a Haake Universal Temperature Controler System (UTC) 6001 temperature control system.

An anti-evaporation bell jar was positioned above the plane of the cone and of the plate during the measurement.

The temperature of the gap was set at 25±0.5° C. An isotherm of 120 seconds at this temperature was applied before each measurement.

An upward oscillation curve with an amplitude sweep was imposed with a stress ramp ranging from 10⁻³ to 10³ Pa, with a frequency set at 1 Hz with 50 stationary phases.

The rheological data of the 7 formulae are described in the table below.

TABLE 2

| | | Ex. 2 | Ex. 3 | Ex. 4 |
|---|---|---|---|---|
| | Ex. 1 | outside the | outside the | outside the |
| Measurements of the rheological behaviour | invention | invention | invention | invention |
| Yield point stress (T₀ in Pa) | 8 | 20 | 20 | 20 |
| Consistency factor k | 45 | 60 | 60 | 60 |
| Flow index n | 0.3 | 0.3 | 0.3 | 0.3 |
| Viscosity at 0.1 s⁻¹ (in Pa · s) | 100 | 280 | 250 | 200 |
| Viscosity at 1 s⁻¹ (in Pa · s) | 20 | 40 | 40 | 35 |
| Viscosity at 10 s⁻¹ (in Pa · s) | 7 | 10 | 9 | 9 |
| Viscosity at 100 s⁻¹ (in Pa · s) | 1.7 | 2.5 | 2.5 | 2.0 |
| Viscosity at 1000 s⁻¹ (in Pa · s) | 0.36 | 0.5 | 0.48 | 0.44 |

As the viscosity is not constant whatever the value of the rate gradient, the compositions of Examples 1 to 4 are non-newtonian (non-ideal) fluids. Rheological Herschel-Bulkley modelling of equation:

$$T = {T}_{0} + {k ( γ )}^{n} \qquad [ Math 1 ]$$

was applied to the rheological curves of the formulae 1 to 4 studied: this resulted in a correlation r²>0.995.

For non-ideal fluids, certain parameters were identified:
the shape of the curve showed a more or less shear-thinning nature (characterized by the index n obtained by modelling)
the yield point To (point starting from which the formulation flowed and its viscosity decreased)
the consistency factor k
the viscosity as a function of the rate gradient.

The values of the yield point To, of the consistency factor k and also the viscosity values obtained with Example 1 of the invention comprising dodecamethylpentasiloxane are significantly lower than those obtained with Example 2 outside the invention without volatile silicone oil, Example 3 outside the invention comprising the cyclic silicone oil cyclopentasiloxane and Example 4 comprising a dimethicone 6 cSt. These results showed that Example 1 of the invention exhibits a more fluid texture making it possible to be easily taken up and spread on the skin.

1. Anhydrous composition for caring for and/or making up keratin materials, in particular the skin, comprising, preferably in a physiologically acceptable medium:
a) an oily phase comprising
i) at least one volatile hydrocarbon oil H₁ and
ii) dodecamethylpentasiloxane; and
b) at least one silicone elastomer possessing carboxylic acid functions.

2. Composition according to claim 1, where the at least one volatile hydrocarbon oil H₁ is chosen from C₈-C₁₆ isoalkanes.

3. Composition according to claim 1, where the at least one volatile hydrocarbon oil H₁ is chosen from volatile linear alkanes comprising from 6 to 14 carbon atoms.

4. Composition according to claim 1, where the at least one volatile hydrocarbon oil H₁ is:
a mixture of n-dodecane and of n-tetradecane;
a mixture of volatile linear C₉-C₁₂ alkanes; or
a mixture of n-undecane (C₁₁) and of n-tridecane (C₁₃).

5. Composition according to claim 1, where the at least one volatile hydrocarbon oil(s) H₁ is (are) present in contents ranging from 50% to 100% by weight with respect to the total weight of the oily phase.

6. Composition according to claim 1, where the at least one volatile hydrocarbon oil(s) H₁ is (are) present in contents ranging from 50% to 95% by weight with respect to the total weight of the composition.

7. Composition according claim 1, where the oily phase is present in an amount ranging from 50% to 95% by weight with respect to the total weight of the composition.

8. Composition according to claim 1, wherein the at least one silicone elastomer possessing carboxylic acid functions is present in an amount from 2% to 25% by weight; with respect to the total weight of the composition.

9. Composition according to claim 1, where the dodecamethylpentasiloxane is present in an amount from 1% to 50% by weight with respect to the total weight of the oily phase.

10. Composition according to claim 1, where the dodecamethylpentasiloxane is present in an amount from 0.5% to 25% by weight with respect to the total weight of the composition.

11. Composition according to claim 1, where the at least one silicone elastomer possessing carboxylic acid functions is chosen from at least one of the compounds corresponding to the following formula (I): 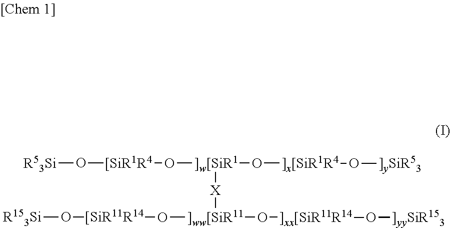
in which the radical X corresponds to the following formula (i) or to the following formula (i′): 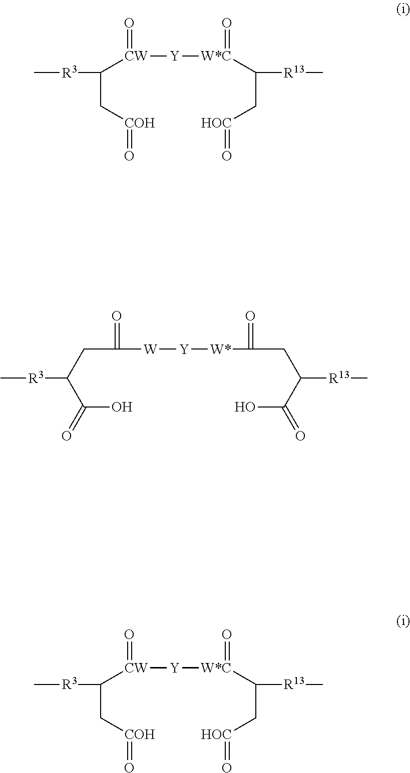
in which
W and W* independently denote an oxygen atom (O) or an N—R group where each R radical independently denotes a hydrogen atom (H) or an R¹ radical;
Y is a divalent group;
R¹, R¹¹, R⁴, R¹⁴, R⁵ and R¹⁵ independently denote a substituted or unsubstituted hydrocarbyl group;
R³ and R¹³ independently denote a divalent group;
w and ww are independently integers ranging from 0 to 1000;
x and xx are independently integers ranging from 1 to 100; and
y and yy are independently integers ranging from 0 to 1000.

12. Composition according to claim 1, where the at least one silicone elastomer possessing carboxylic acid functions is chosen from at least one of the following compounds 1 to 12:

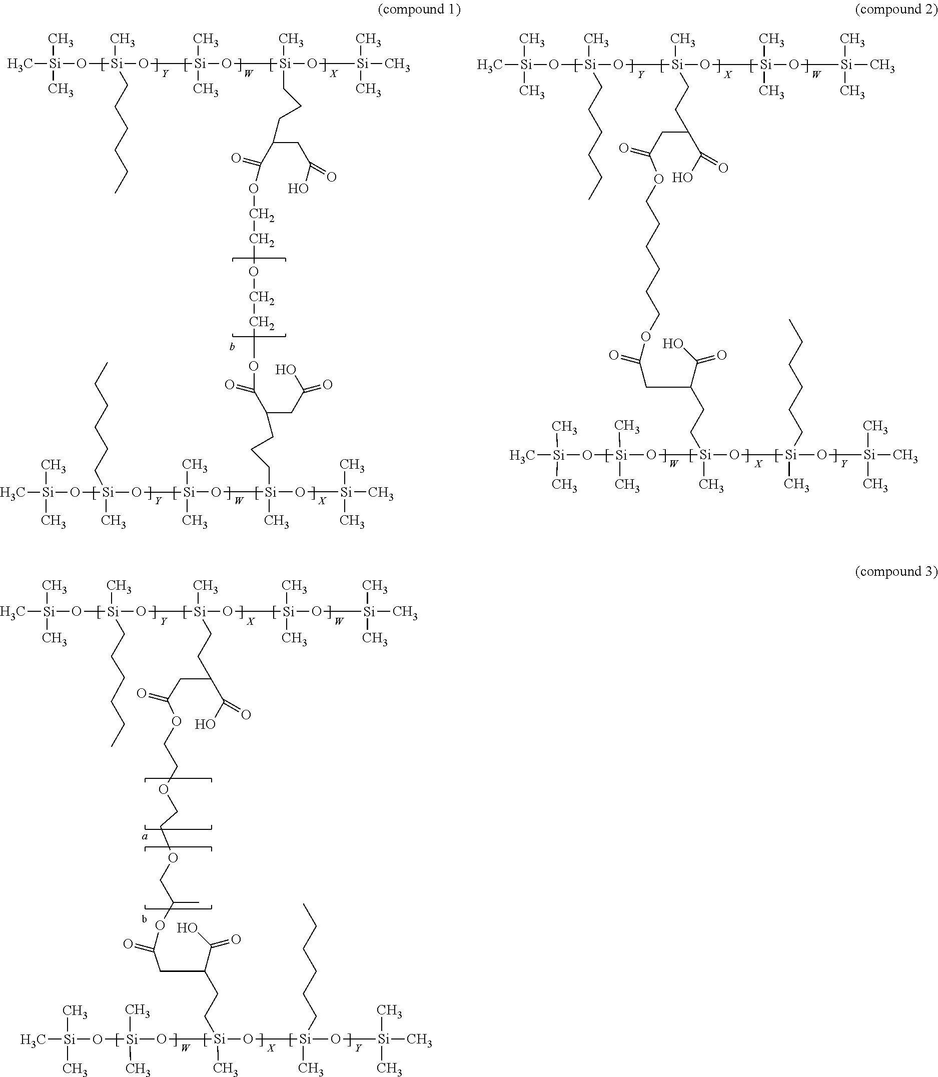

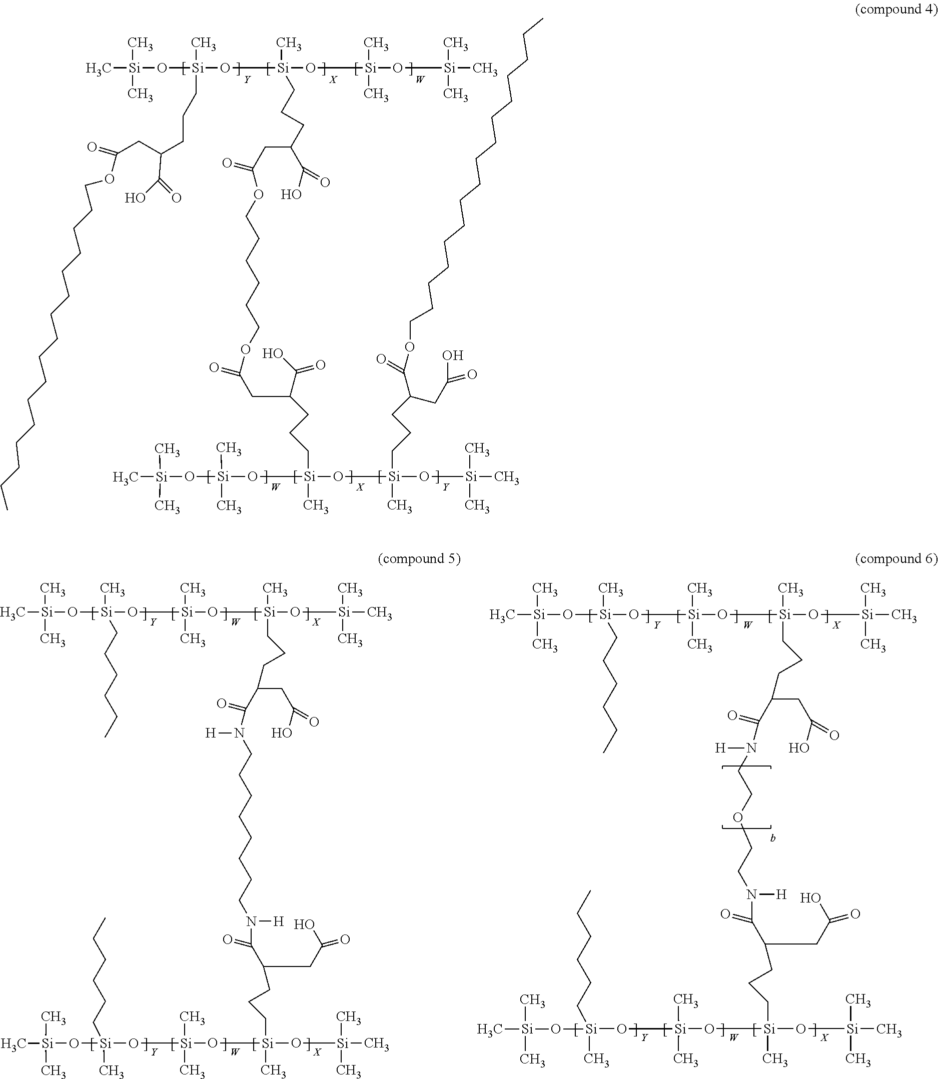

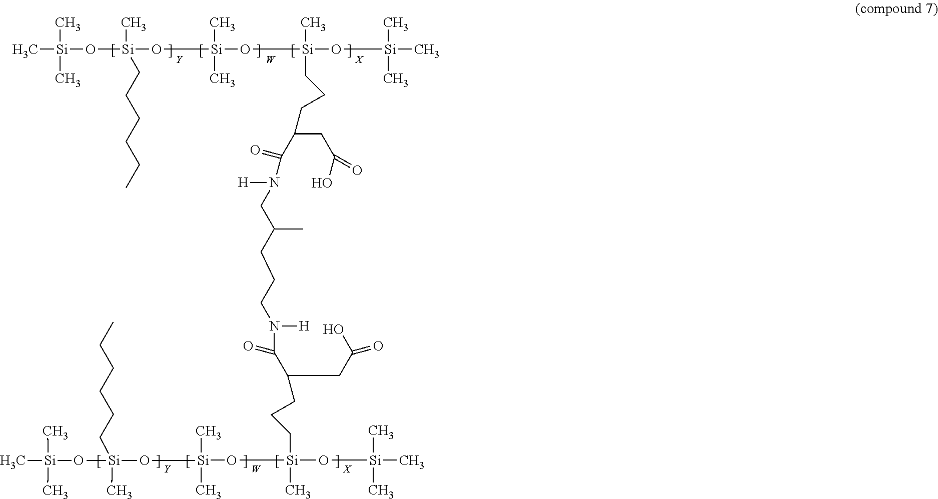

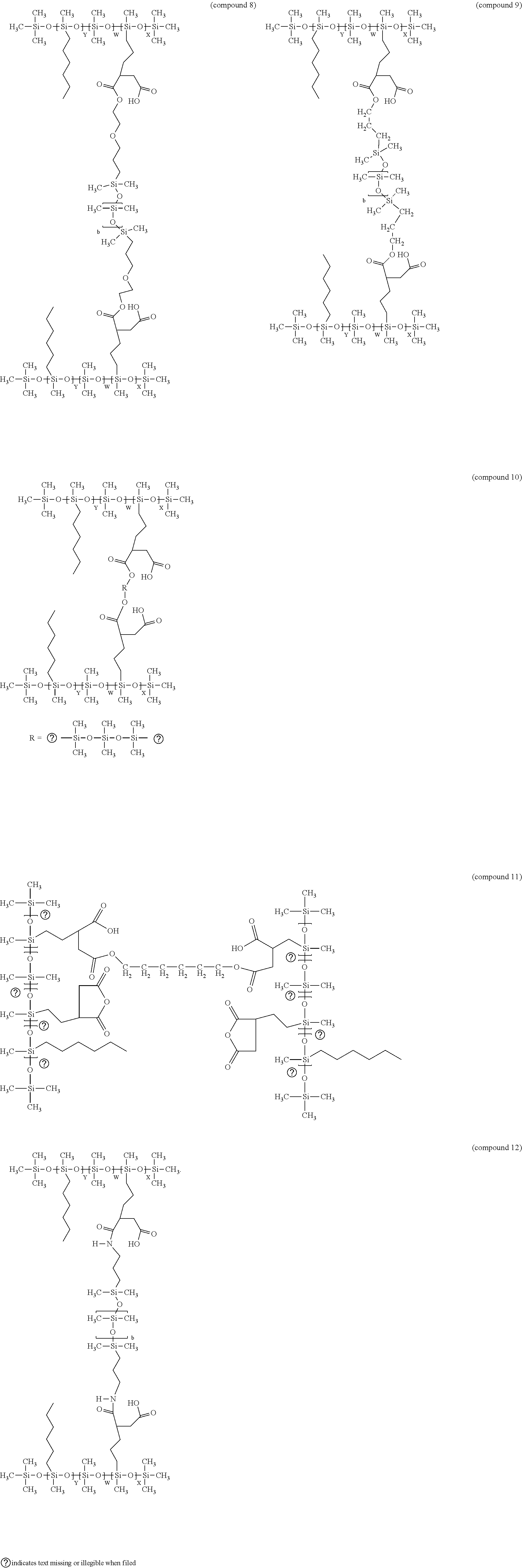

13. Composition according to claim 12, where the at least one silicone elastomer possessing carboxylic acid functions is the compound 10 and/or the compound 11.

14. Composition according to claim 1, where the at least one silicone elastomer possessing carboxylic acid functions is Hexyl/Succinyl Dimethicone Crosspolymer.

15. Composition according to claim 1, where the at least one silicone elastomer possessing carboxylic acid functions is in dispersion in at least one oil H₂ and in gel form.

16. Composition according to claim 15, where the at least one oil H₂ is a volatile hydrocarbon oil chosen from —C₈-C₁₆ isoalkanes and linear alkanes comprising from 6 to 14 carbon atoms.

17. Composition according to claim 15, where the at least one oil H₂ and the at least one oil H₁ are identical.

18. Composition according to claim 15, where the dispersion of silicone elastomer possessing carboxylic acid functions comprises from 10% to 40% by weight of active material of silicone elastomer with respect to the total weight of the dispersion.

19. Composition according to claim 15, where the at least one silicone elastomer possessing carboxylic acid functions is in dispersion in isododecane.

20. Composition according to claim 13, where the at least one silicone elastomer possessing carboxylic acid functions is present in isododecane at a content of active material of 30% by weight with respect to the total weight of the dispersion.

21. Composition according to claim 14, where the Hexyl/Succinyl Dimethicone Crosspolymer is in the form of a gel in dispersion in isododecane.

22. Composition according to claim 1, additionally comprising at least one pulverulent colorant.

23. Composition according to claim 22, where the at least one pulverulent colorant(s) is (are) present in a content from 5% to 40% by weight, with respect to the total weight of the composition.

24. Composition according to claim 1, additionally comprising at least one additive chosen from UV sunscreens, C₂-C₄ monoalcohols, moisturizing agents, fat-soluble colorants, lipophilic thickening or gelling agents, preservatives, fragrances, and their mixtures.

25. Method for coating keratin materials, more particularly for making up and/or caring for keratin materials, such as the skin, characterized in that it comprises the application to keratin materials of a composition as defined in claim 1.
